(19) United States
(12) Patent Application Publication
(10) Pub. No.: US 2021/0259675 A1
(43) Pub. Date: Aug. 26, 2021

(54) OROGASTRIC TUBE GUIDE

(71) Applicant: H. LEE MOFFITT CANCER CENTER AND RESEARCH INSTITUTE, INC., Tampa, FL (US)

(72) Inventors: Tariq Chaudhry, Tampa, FL (US); Mario Kikic, Riverview, FL (US)

(21) Appl. No.: 17/315,953

(22) Filed: May 10, 2021

Related U.S. Application Data
(63) Continuation of application No. 15/022,886, filed on Mar. 17, 2016, now abandoned.
(60) Provisional application No. 61/878,832, filed on Sep. 17, 2013.

(51) Int. Cl. A61B 17/02; A61M 16/04

(52) U.S. Cl. CPC A61B 17/0218; A61M 16/0493; A61M 16/0484; A61M 2025/022; A61M 16/0495; A61M 16/0463; A61M 16/0488

(57) ABSTRACT

Provided is an orogastric tube guide that can be used to accurately and quickly place an orogastric tube into the esophagus of a subject. In some embodiments, the orogastric tube guide is placed into the mouth of the subject after the subject has been intubated with an endotracheal tube. When placed in the midline of the subject's mouth, the disclosed orogastric tube guide aims placement of an orogastric tube towards the subject's esophagus. The orogastric tube guide also optionally contains a bite block to prevent a patient's teeth from clamping down on an endotracheal tube. The orogastric tube guide also optionally contains ports that can be attached to air tubing for providing supplemental oxygen after endotracheal extubation, thereby avoiding the need for nasal cannula.

### CROSS-REFERENCE TO RELATED APPLICATIONS

This application claims benefit of U.S. Provisional Application No. 61/878,832, filed Sep. 17, 2013, which is hereby incorporated herein by reference in its entirety.

### BACKGROUND

Orogastric tubes are used during surgery to aspirate gastric contents and reduce postoperative nausea and vomiting. The orogastric tubes are inserted after intubation with endotracheal tubes. However, this is a blind procedure, often necessitating multiple attempts before successful intubation. This can lead to cross-contamination from gastric secretions and damage to the patient's mucosa. In addition, after several failed attempts, orogastric tubes must be discarded, leading to unnecessary waste.

### SUMMARY

Provided is an orogastric tube guide that can be used to accurately and quickly place an orogastric tube through the oropharynx into the esophagus of a subject. In some embodiments, the orogastric tube guide is placed into the mouth of the subject after the subject has been intubated with an endotracheal tube. When placed in subject's mouth along the midline, the disclosed orogastric tube guide aims towards the subject's esophagus facilitating quick and accurate placement of an orogastric tube. The orogastric tube guide can also contain a bite block to prevent a patient's teeth from clamping down on an endotracheal tube. The orogastric tube guide also optionally contains ports that can be attached to air tubing for providing supplemental oxygen after endotracheal extubation, thereby avoiding the need for nasal cannula.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of an embodiment of an orogastric tube guide.

FIG. 2 is a cross-section view of an embodiment of an orogastric tube guide.

FIG. 3 is a side view of an embodiment of an orogastric tube guide.

FIG. 4 is top-down view of an embodiment of an orogastric tube guide.

FIG. 5 is a perspective view of an embodiment of an orogastric tube guide assembly with oxygen and carbon dioxide tube connections.

FIG. 6 is an image of an embodiment of an orogastric tube guide.

FIG. 7 is an image of an embodiment of an orogastric tube guide assembly with oxygen and carbon dioxide tube connections placed in the mouth of a manikin.

FIG. 8 shows example measurements of an embodiment of an orogastric tube guide.

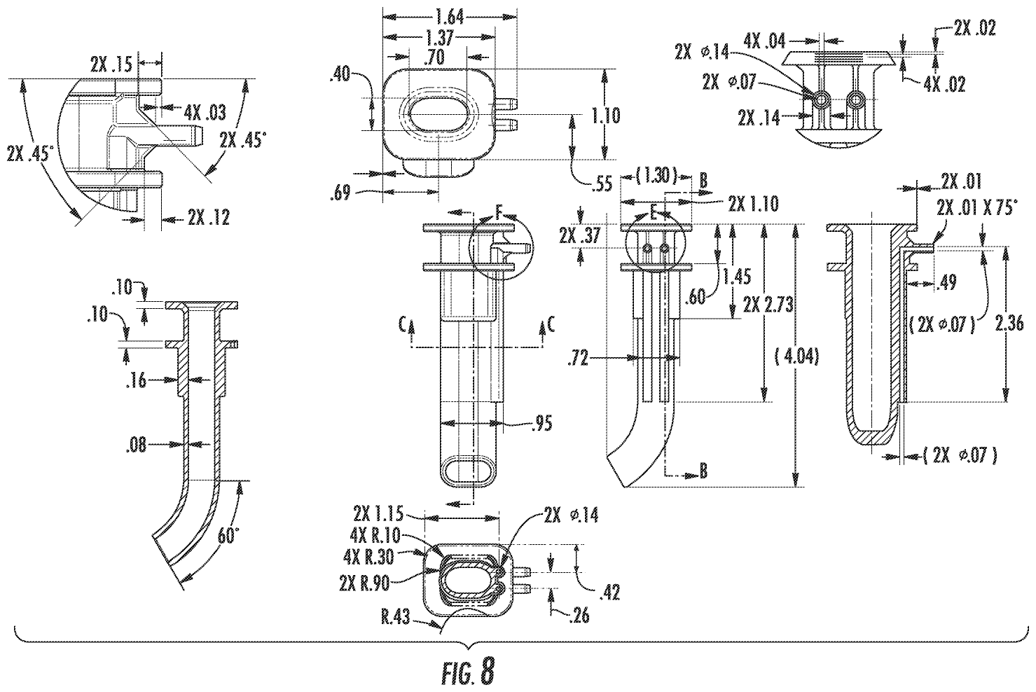

### DETAILED DESCRIPTION

The present invention now will be described more fully hereinafter with reference to specific embodiments of the invention. The invention can be embodied in many different forms and should not be construed as limited to the embodiments set forth herein; rather, these embodiments are provided so that this disclosure will satisfy applicable legal requirements.

As used in the specification, and in the appended claims, the singular forms “a,” “an,” “the,” include plural referents unless the context clearly dictates otherwise.

The term “comprising” and variations thereof as used herein are used synonymously with the term “including” and variations thereof and are open, non-limiting terms.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

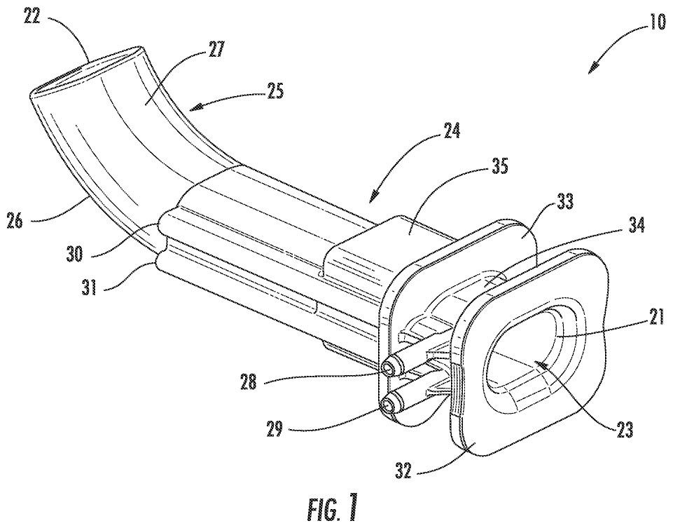

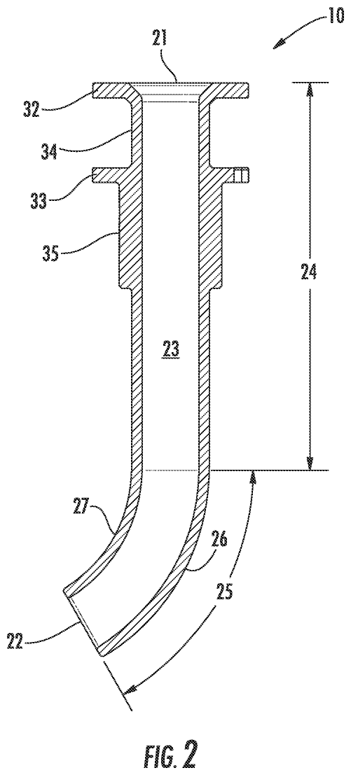

Now referring more particularly to FIGS. 1 to 6 of the drawings, an orogastric tube guide 10 is provided. As shown in FIGS. 1 and 2, the disclosed orogastric tube guide 10 has a distal end 21, a proximal end 22, and a lumen 23 that extends continuously from the distal end 21 to the proximal end 22. The cross-section of the lumen 23 is preferably elliptical with a minimum and maximum diameter. The lumen 23 preferably has a minimum diameter throughout its length large enough to accommodate an orogastric tube (e.g., 24 French to 42 French) and a temperature probe to be inserted through the lumen 23. For example, the lumen can have a minimum diameter of about 0.20 inches to about 0.6 inches, such as about 0.40 inches.

The disclosed orogastric tube guide 10 can have a straight portion 24 that extends from the distal end 21 towards the proximal end 22 of the tube guided 10, and a curved portion 25 extending from the end of the straight portion 24 to the proximal end 22 of the tube guide 10. The curved portion 25 has a convex surface 26 and a concave surface 27. The convex surface 26 can rest along the back of the subject's throat and direct the orogastric tube down towards the esophagus. In some embodiments, the curved portion 25 has an angle relative to the straight portion 24 of about 40 to 80 degrees. In particular, the curved portion 25 can have an angle of about 60 degrees.

The orogastric tube guide 10 also optionally has ports 28, 29 that can be attached to air tubing. Each port 28, 29 is fluidly connected to channels that extend along the straight portion 24 toward the proximal end 22 of the tube guide 10. The channels terminate at vents 30, 31 positioned for air transfer to the back of the subject's throat. These ports 28, 29 and vents 30, 31 have the advantage of providing supplemental oxygen after endotracheal extubation, thereby avoiding the need for nasal cannula.

The orogastric tube guide 10 also optionally has flanges 32, 33 near the distal end 21 for gripping and advancing the tube guide 10. As shown in FIG. 1, the tube guide 10 can have a first flange 32 positioned at the distal end 21 of the tube guide 10. The tube guide 10 can also have a second flange 33 positioned proximally to the first flange 32. The portion of the tube guide between the first flange 32 and second flange 33 defines a gripping portion 34. This gripping portion 34 is preferably sized to allow fingers to be positioned between the first flange 32 and second flange 33. As shown in FIG. 1, the ports 28, 29 can be positioned in this gripping portion 34 between the first 32 and second 33 flanges.

The disclosed orogastric tube guide 10 also optionally contains a bite block 35. A bite block prevent a patient's teeth from clamping down on an endotracheal tube, thus pinching the tube and restricting or entirely cutting off the flow of oxygen or air to the patient's lungs through the endotracheal tube. Reduction of the flow of air or oxygen to such a patient in an emergency situation may critically impair chances for the patient's recovery. Various bite blocks are known in the art but generally are complicated in design and difficult and costly to make and/or difficult to assemble and connect to an endotracheal tube. Preferably, the bite block 35 is sized larger than the endotracheal tube. For example, if the outer diameter of the straight portion 24 is not larger than that of the endotracheal tube, the bite block 35 can optionally be a raised portion as shown in FIG. 1. However, the tube guide 10 can in some embodiments have an outer diameter larger than the endotracheal tube so that a raised portion is not necessary.

The disclosed orogastric tube guide 10 preferably comprises a rigid material, such as a metal or plastic. Optionally, the orogastric tube guide 10 can be sterilized, for example by chemical and/or heat based techniques.

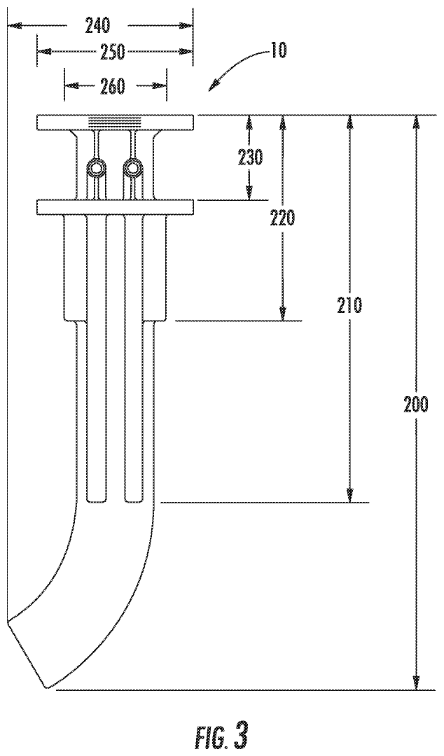

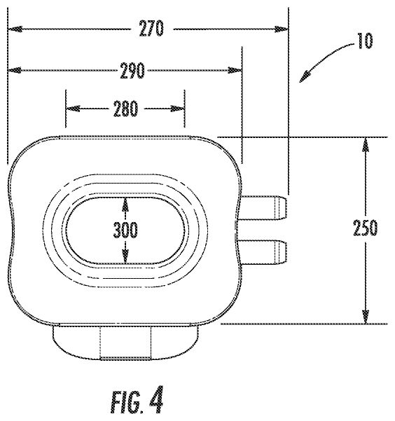

FIGS. 3 and 4 illustrate dimensions of the orogastric tube guide 10. The distance 200 between the distal end 21 and the proximal end 22 along the convex surface 26 can be about 1 to about 5 inches, adjusted smaller or larger if it is intended for use in a child or large adult, respectively. For example, the distance 200 between the distal end 21 and the proximal end 22 along the convex surface 26 can be about 1 to 3 inches for children and about 3 to 5 inches for adults. The distance 210 of the straight portion 24 can be about 2.7 inches. The distance 220 between the distal end 21 of the tube guide 10 and the proximal end of the bite block 35 can be about 1.45 inches. The distance 230 between the first flange 32 and second flange 33 can be about 0.60 inches. The outer diameter 260 of the bite block 35 can be about 0.72 inches. The distance 240 can be about 1.30 inches. The first flange 32 and/or second flange 33 can have a width 290 of about 1.37 inches and a height 250 of about 1.10 inches. The distance 270 can be about 1.64 inches. The lumen 23 can have a maximum diameter 280 of about 0.70 inches and a minimum diameter 300 of about 0.2 to about 0.5 inches, including about 0.40 inches.

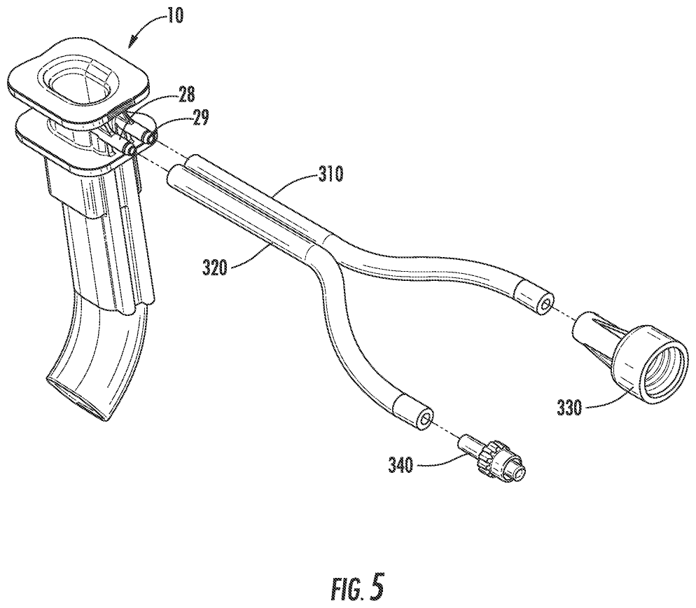

FIG. 5 illustrates an example orogastric tube guide 10 assembled with oxygen and carbon dioxide tube connections. In FIG. 5, tubing 310 is connecting port 28 to a threaded oxygen connector 330; and tubing 320 is connecting port 27 to a carbon dioxide Leur fitting 340. It is understood that ports 27 and 28 are interchangeable.

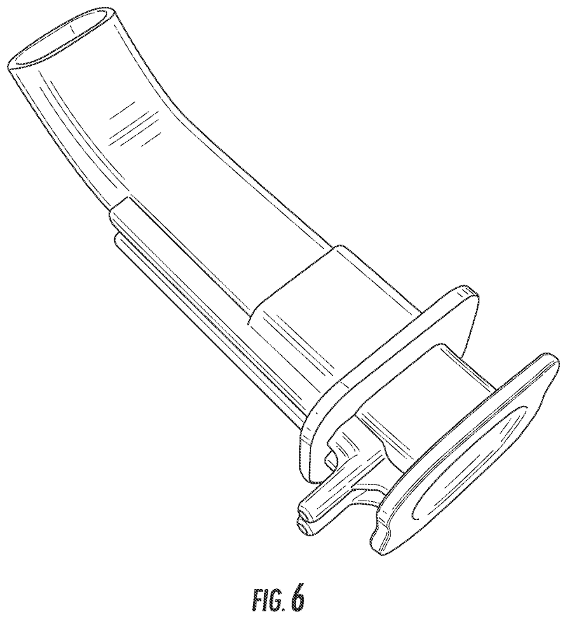

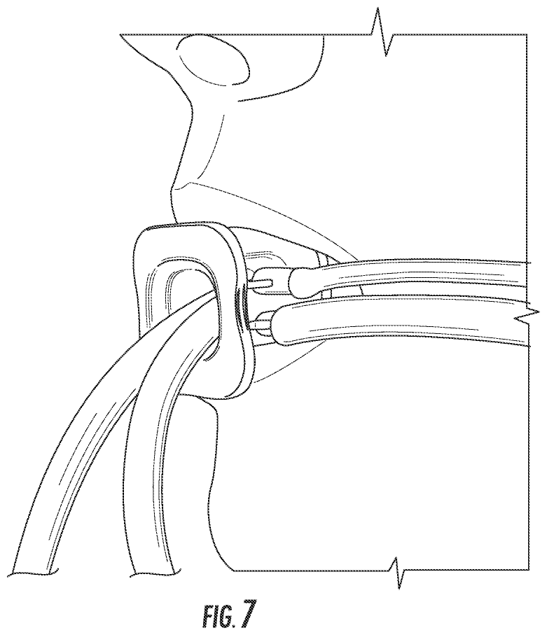

An example orogastric tube guide 10 is shown in FIGS. 6 and 7. In FIG. 7, orogastric tubes are shown inserted through the lumen 23, and air tubing 310, 320 are connected to the ports 27, 28.

A number of embodiments of the invention have been described. Nevertheless, it will be understood that various modifications may be made without departing from the spirit and scope of the invention.

Disclosed are materials, systems, devices, compositions, and components that can be used for, can be used in conjunction with, can be used in preparation for, or are products of the disclosed methods, systems and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein.

1. An orogastric tube guide, comprising a tube having a distal end, a proximal end, and a lumen that extends continuously from the distal end to the proximal end, wherein the tube comprises:
a straight portion extending proximally from the distal end; and
a curved portion extending proximally from the straight portion, wherein the curved portion has an angle of about 40 degrees to about 80 degrees relative to the straight portion.

2. The orogastric tube guide of claim 1, wherein the curved portion comprises a convex surface and a concave surface, wherein the convex surface rests along the back of a subject's throat and directs an orogastric tube down towards the subject's esophagus.

3. The orogastric tube guide of claim 1, wherein the lumen has an elliptical cross-section.

4. The orogastric tube guide of claim 1, further comprising one or more ports that can be connected to air tubing fluidly connected to a lumen that extends proximally along the straight portion of the tube and terminates at one or more vents.

5. The orogastric tube guide of claim 1, further comprising one or more flanges near the distal end for gripping and advancing the orogastric tube guide into the mouth of a subject.

6. The orogastric tube guide of claim 5, comprising a first flange positioned at the distal end of the tube and a second flange positioned proximally to the first flange.

7. The orogastric tube guide of claim 6, wherein the portion of the tube between the first flange and second flange defines a gripping portion, wherein the gripping portion is sized to allow a finger to be positioned between the first flange and second flange.

8. The orogastric tube guide of claim 1, further comprising a bite block along the straight portion with an outer diameter larger than an endotracheal tube.

9. The orogastric tube guide of claim 1, wherein the distance between the distal end and the proximal end is about 1 to about 5 inches.

10. The orogastric tube guide of claim 1, wherein length of the straight portion is about 2.7 inches.

11. The orogastric tube guide of claim 1, wherein the lumen comprises a minimum diameter throughout its length large enough to accommodate an orogastric tube.

12. The orogastric tube guide of claim 11, wherein the orogastric tube has a 24 French to 42 French gauge.

13. The orogastric tube guide of claim 11 or 12, wherein the lumen is elliptical and has a minimum diameter of about 0.20 inches to about 0.6 inches.

14. The orogastric tube guide of claim 13, wherein the lumen has a minimum diameter of about 0.2 inches to about 0.50 inches.

15. The orogastric tube guide of claim 1, wherein the curved portion has an angle of about 60 degrees relative to the straight portion.
